(19) United States
(12) Patent Application Publication
(10) Pub. No.: US 2009/0171199 A1
(43) Pub. Date: Jul. 2, 2009

(54) METHOD AND INSTRUMENT AS WELL AS USE OF INSTRUMENT FOR PERMANENT APPLICATION OF REFERENCE MARKER FOR PRECISION AIMING OF TREATMENT BEAM

(76) Inventors: Anders Widmark, Umea (SE); Per-Olov Lofroth, Umea (SE); Per Bergstrom, Umea (SE)

Correspondence Address: TAROLLI, SUNDHEIM, COVELL & TUMMINO L.L.P., 1300 EAST NINTH STREET, SUITE 1700, CLEVEVLAND, OH, 44114, US

(21) Appl. No.: 12/278,059

(22) PCT Filed: Jan. 31, 2007
(86) PCT No.: PCT/SE07/00094

§ 371 Date: Feb. 9, 2009

(30) Foreign Application Priority Data
Feb. 3, 2006 (SE) 0600237-2

(51) Int. Cl. A61M 37/00

(52) U.S. Cl. 600/433

(57) ABSTRACT

A method for permanently applying via the urethra a reference marker in the prostate gland in order by means of the marker to allow precision aiming of a treatment beam at a cancer tumor therein, whereby a catheter comprising a duct and a reference marker disposed for movement in the duct is introduced into the urethra and inserted through it to the prostate gland, a device, with guidance by the reference marker, is caused to, from the duct, penetrate the catheter and/or a membrane totally or partly covering the catheter and is inserted in glandular tissue surrounding the catheter and the urethra, and the marker is thereafter moved by a means along the penetrating and guiding means and is driven by the movement means, with guidance by the penetrating and guiding means, into said glandular tissue for permanent application therein. An instrument for this purpose comprises a catheter (9) with a duct (11), a reference marker (7) disposed in the duct, a means (12) for indicated penetration and guidance, and a means (14) for moving the reference marker. Said instrument may also be used for permanent application of a reference marker in other tissue.

The present invention relates to a method and an instrument for permanently applying via the urethra at least one reference marker in the prostate gland in order by means of the marker to enable precision aiming of a treatment beam at a cancer tumour in the prostate gland. The invention also relates to the use of said instrument for permanent application of at least one reference marker in other tissue situated around a body cavity, body orifice etc. than prostate gland tissue.

During the last forty years, radiation treatment of localised prostate cancer has been used as one form of treatment among others. The radiation doses considered suitable for this treatment have been within the range 65 to 70 Gy. However, some cancer cells have been found to survive such a treatment dose. One way of dealing with such residual cells is to increase the treatment dose, by so-called dose scaling, primarily to the range 76 to 80 Gy or more. Owing to precision limitations of conventional technology, such radiation treatment entails a greater risk of affecting adjacent organs. Around the prostate gland, this greater risk mainly involves side effects in the form of damage to the rectum and the urinary bladder, since they are usually within the radiation field. The risk of damage is particularly great at higher radiation doses.

The preparations prior to radiation treatment of prostate cancer may be briefly described as follows:

The patient undergoes computer tomography to determine the geometry and position of the prostate gland. The position of the prostate gland is indicated by marking the patient's skin. The markings thereafter serve as aiming points for the treatment beams.

Even if the geometry of the prostate gland is known, radiation treatment involves problems in that the position of the prostate gland is not constant relative to the pelvis, nor in relation to the skin where the markings are situated. The patient's subcutaneous fat may cause the markings on the skin to move relative to the pelvis, and various muscular tensions etc. in the region round the prostate gland may move the latter upwards by a centimetre relative to the pelvis. Variations in the fullness of the rectum and bladder may also affect the position of the prostate gland. All this means that the radiation field has to include a margin of 1.5 to 2 cm around the determined position of the prostate gland, resulting in irradiation of the rectum and the urinary bladder. The possibility of using very high radiation doses is therefore limited.

The method for precision aiming of a radiation beam at an internal cancer tumour in a patient is distinguished, or more correctly preceded, by at least one reference marker being introduced into the patient to a determined position relative to the cancer tumour, the geometry of which is determined, and by the treatment being planned relative to the reference marker. The geometric determination of the tumour may with advantage involve not only computer tomography (“slice” radiography) but also the use of magnetic imaging or other suitable radiological techniques, and the reference marker may for example take the form of contrast fluid, gold, tantalum, tungsten or some other material which shows up under computer tomography, magnetic imaging or similar radiological techniques.

The method also entails the patient, at the commencement of treatment, being in a conventional manner placed in a treatment posture, and a certain point in the beam cross-section being defined by an aiming means. The geometric determination and the description of the cancer tumour are effected on the basis of one or more reference markers, and the resulting information provides the basis for constructing the treatment beam cross-section and for positioning the aiming means in the beam cross-section with advantage so that, when the beam is aligned with the cancer tumour, the positions of the aiming means and of the reference marker in the beam cross-section coincide despite being at a distance from one another.

The method also involves the patient being irradiated with said at least one reference marker at the determined location, the mutual positioning of the reference marker or markers and the aiming means being read on the basis of the beam passing through the patient, and the mutual positioning being adjusted as necessary in order to ensure precision aiming of the beam at the cancer tumour before the patient is given the remaining dose of the treatment session. Using the beam to project the location of the aiming means and the location(s) of the reference marker(s) on a sensitive surface or detector provides a display of their mutual positioning. A method as above is described in, for example, patent specification SE 9700984-9.

A disadvantage of the arrangement for implementing the above-mentioned method is that the catheter for introducing at least one reference marker, and hence also the reference marker, have to be removed from the urethra after the treatment. On the occasion of each further treatment, a catheter with one or more reference markers has again to be introduced into the urethra or permanent markers have to be implanted in the prostate via needles in the perineum or rectum.

The object of the present invention is therefore to propose a method and an instrument for permanent application of one or more reference markers in the prostate gland via the urethra. Such an instrument has to be as small as possible and easy to handle and its insertion has to involve no problems. Another object is to propose also using such an instrument for permanent application of reference markers in other tissues.

The objects indicated are in principle achieved by a method with the characterising steps indicated in claim 1, by an instrument with the characterising features indicated in claim 8, and by use according to claim 34.

The invention provides an easy and reliable way of using an appropriate instrument without ultrasound or other diagnostic guidance to apply one or more reference markers permanently in an organ or other tissue so that the reference marker(s) always assume(s) the same position(s) therein. It thus becomes possible to repeatably aim the treatment beam with precision relative to said at least one reference marker so that the beam hits the cancer tumour with precision. It is also possible, as previously, to monitor, correct and verify that the cancer tumour really is in the path of the treatment beam, and this can be done on the occasion of each treatment.

EP 1 380 319 B1, for example, describes a device which likewise involves a catheter with at least one “reference marker”, a penetrating and guiding means disposed for movement in the catheter, and a movement means for moving the “reference marker”, but the penetrating and guiding means takes the form of a tube with the “reference marker” applied in and accompanying the tube during penetration of the “envelope” and surrounding glandular tissue.

The fact that the penetrating and guiding means of the instrument according to the present invention is instead guided by the reference marker during penetration of the membrane and surrounding glandular tissue not only results in the provision of guidance but also makes it possible for the penetrating and guiding means to be made smaller and of simpler design, with a reference marker directly in the catheter duct instead of in a tube in a duct in a catheter. Another difference is of course that the reference marker does not accompany the penetrating and guiding means during said penetration but is inserted later, with guidance by the penetrating and guiding means.

Further features and advantages of the invention are indicated by the detailed description set out below of a preferred version of it, which serves as an example and therefore does not limit the extent of protection of the invention. The description is with reference to the attached drawings, in which

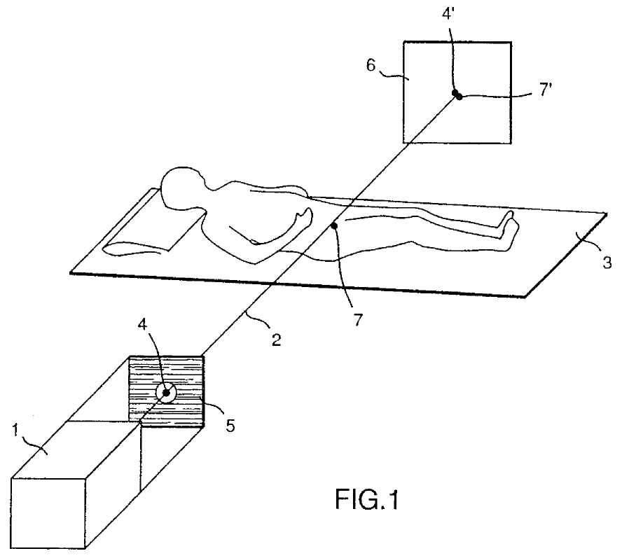

FIG. 1 depicts schematically an arrangement for precision aiming of a treatment beam after application of a reference marker;

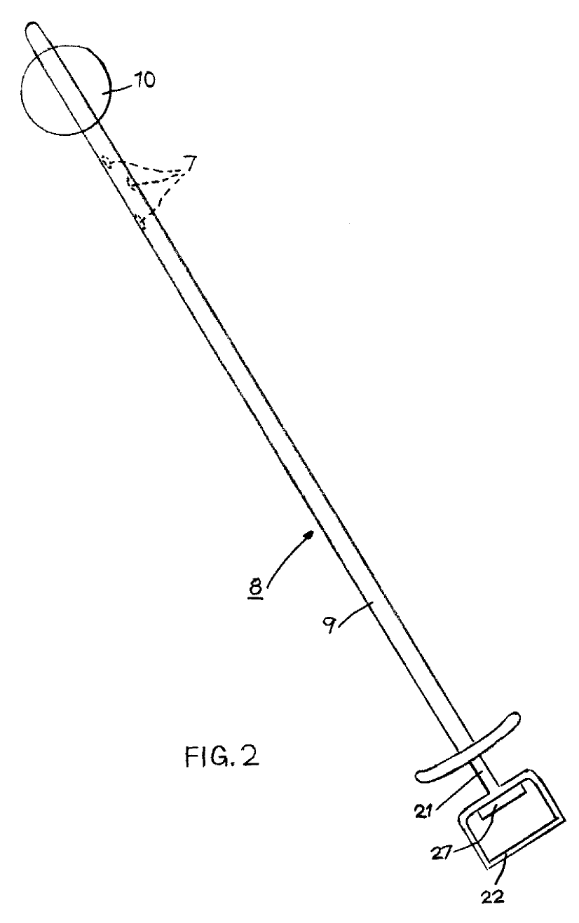

FIG. 2 depicts schematically an instrument according to the invention for permanent application of at least one reference marker;

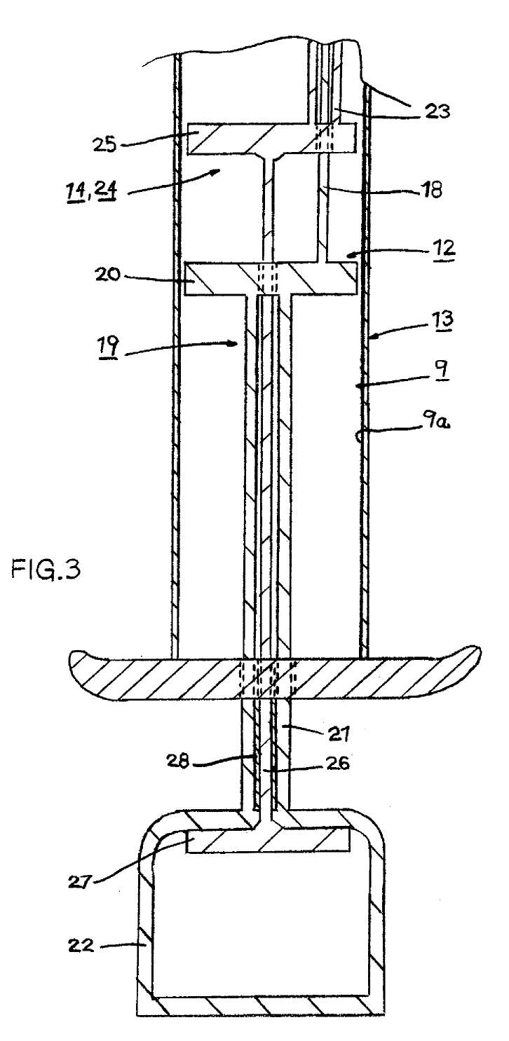

FIG. 3 is a schematic enlarged longitudinal section through a portion of the instrument according to FIG. 2; and

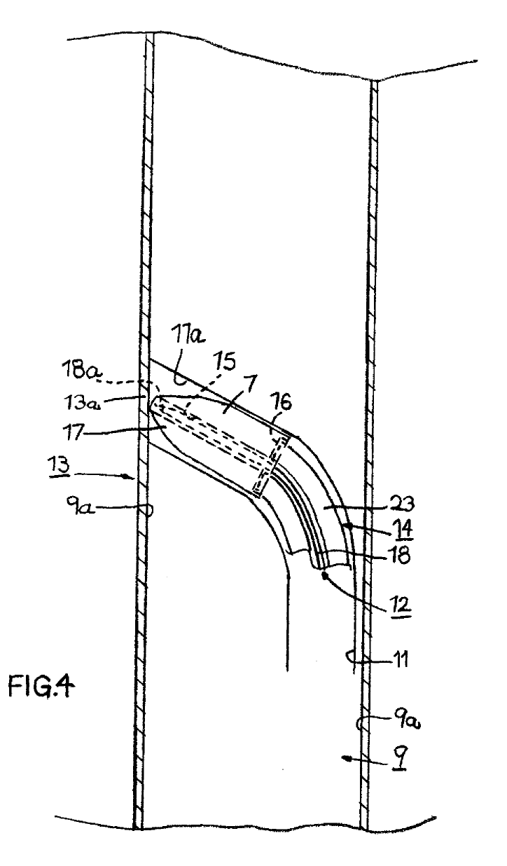

FIG. 4 is a schematic still more enlarged longitudinal section through another portion of the instrument according to FIG. 2.

FIG. 1 thus depicts an arrangement for implementing the known method described above for precision aiming of a treatment beam at a cancer tumour, here a tumour in the prostate gland, according to patent specification SE 9700984-9. The arrangement comprises a conventional treatment appliance 1 for emitting a treatment beam 2, and a means 3 on which the patient being treated rests. The patient has at least one reference marker 7 relative to which the location of the cancer tumour is determined. An aiming means 4 disposed in the path of the treatment beam 2 defines a point in the beam cross-section 5. A means 6 reading the treatment beam 2 receives the beam passing through the patient and displays the location of said reference marker 7′ and the location of the aiming means 4′, thereby determining the position of the treatment beam 2 relative to the cancer tumour. A more specific configuration and function of the arrangement are described in more detail in said patent application.

FIG. 2 depicts an instrument 8 for permanent application of at least one reference marker 7 in the prostate gland to enable said precision aiming of the treatment beam 2 at the cancer tumour therein.

The instrument 8 comprises according to the present invention a catheter 9 insertable to the prostate gland via the urethra. The catheter 9 is with advantage introduced far enough into the urethra for its free end, the insertion end, to enter the urinary bladder. There it is possible for a fixing means 10, preferably an expandable balloon disposed at the insertion end, to be activated in order to fix the catheter 9 relative to the urethra and hence also relative to the prostate gland. The catheter 9 is drawn back somewhat and causes the balloon 10, expanded in the version depicted, to abut against the bottom of the urinary bladder at the connection to the urethra. As well as said expandable balloon, the fixing means 10 for the catheter 9 may also comprise, for example, extendable wings, hooks or clips of various kinds which allow the catheter to be released without problems after the reference marker 7 has been applied. An end portion of the catheter 9 which is distal relative to the insertion end remains always outside the urethra.

The catheter 9 has running through it in its longitudinal direction in the vicinity of its insertion end a duct 11 for at least one reference marker 7 (FIG. 4). According to the invention, the reference marker 7 is not fixed as in the state of the art but is disposed for movement in the duct 11.

As indicated in FIG. 3 and particularly in FIG. 4, the instrument according to the invention also comprises a means 12 designed to, from the duct 11, penetrate the catheter 9 and/or a membrane 13 totally or partly covering the catheter, to make its way into glandular tissue surrounding the catheter and the urethra, and to guide the movement of the reference marker 7.

The instrument according to the invention finally comprises also a means 14 designed to move the reference marker 7 along said penetrating and guiding means 12 and, with the latter's guidance, to drive the marker into the glandular tissue surrounding the catheter 9 and the urethra in order permanently to apply the marker therein (FIGS. 3 and 4).

According to the invention, the duct 11 in the catheter 9 is therefore closed at its end portion 11a situated nearest to the insertion end of the catheter, either by the catheter itself or by the membrane 13 totally or partly covering the catheter, or by both, so that no undesired body fluids or tissue parts can make their way into the duct during the insertion of the catheter in the urethra. After the insertion, the catheter 9 and/or the membrane 13 are penetrated by the penetrating and guiding means 12 which, thereafter and after entering the glandular tissue, guides the reference marker 7 to a desired location in the glandular tissue when it is moved by the movement means 14 and is thus driven into the glandular tissue.

The end portion 11a of the duct 11 is preferably directed towards a portion of the sidewall 9a of the catheter 9 or, as in the version depicted, towards a side portion 13a of the membrane 13 covering the catheter, or against both said portion of the sidewall 9a and said side portion 13a, depending on the catheter version and on whether a membrane is provided or not. The end portion 11a thus ends immediately within said portion of the sidewall 9a of the catheter 9 and/or immediately within said side portion 13a of the membrane 13 (FIG. 4).

In the preferred version depicted, the reference marker 7 is disposed in the end portion 11a of the duct 11 and is therefore, after the fixing of the catheter 9, already situated with advantage level with the prostate gland, so that the distance which the reference marker has to travel will be as short as possible.

The reference marker 7 has with advantage a diameter of about 1 to 2 mm and the duct 11 will therefore, in the version depicted, have, at least at the end portion 11a, a somewhat larger diameter. The reference marker 7 has also with advantage a length of about 3 mm. The reference marker 7 will thus not occupy much space but will nevertheless be easy to handle and easy to display by means of suitable equipment. The reference marker 7 is also made of high-density material particularly suited to simple and reliable display in a high-energy treatment beam 2. The material of the reference marker 7 has also to be such as to allow the marker to remain permanently in the body without causing adverse tissue reactions. The reference marker 7 is preferably made of gold, platinum or some other high-density material tolerated by the body.

In the version depicted, the reference marker 7 has a duct 15 running through it in its longitudinal direction. This duct 15 is designed to guide the penetrating and guiding means 12 when the latter penetrates the catheter 9 and/or the membrane 13 totally or partly covering the catheter and makes its way into surrounding glandular tissue, and thereafter to guide the reference marker 7 via the penetrating and guiding means inserted in the glandular tissue when the marker is driven into this glandular tissue. The duct 15 runs with advantage coaxially with the central longitudinal axis of the reference marker 7. As the reference marker 7 in the preferred version is situated in the end portion 11a of the duct 11 immediately within the sidewall 9a of the catheter 9 and/or, as depicted, immediately within the side portion 13a of the membrane 13 covering the catheter, and the penetrating and guiding means 12 in the initial position during the insertion of the catheter in the urethra already has at least a forward end portion situated in said duct 15 in the marker 7, the penetration of the catheter and/or the membrane takes place immediately upon activation of the penetrating and guiding means and at a desired location, and the subsequent driving in of the marker also takes place at a desired location in the glandular tissue.

Said guidance functions may also be achieved by some other suitable configuration of the marker 7. The reference marker 7 has a recess 16 at one end to enable the movement means 14 to cooperate effectively with the marker. At the opposite end, the driving-in end, the reference marker 7 narrows to a tip 17 to make it easier to drive into surrounding glandular tissue.

The penetrating and guiding means 12 comprises a flexible penetrating and guiding part 18 which is disposed for movement in the duct 11 in the catheter 9 and which has connected to it a first manoeuvring device 19 by which the penetrating and guiding part is caused to penetrate the catheter and/or the membrane 13 totally or partly covering the catheter and to make its way into the glandular tissue surrounding the catheter and the urethra. The penetrating and guiding part 18 takes the form, in the preferred version, of a thin metal wire with a pointed front end 18a which, as indicated above, is initially situated in the duct 15 in the reference marker 7. The penetrating and guiding part 18 has with advantage a diameter of about 0.3 to 0.5 mm. Said first manoeuvring device 19 takes the form, in the preferred version depicted, of a piston 20 which is disposed for movement in the catheter 9 in the latter's longitudinal direction, is connected to the penetrating and guiding part 18 and communicates, via a piston rod 21, with a manoeuvring part 22, e.g. a handle, disposed externally to the catheter, for moving the piston in the catheter whereby the piston in its turn does of course move the penetrating and guiding part. The manoeuvring part 22 is of course external to the catheter 9 at the latter's aforesaid distal end portion outside the urethra, so that it is readily accessible for activating or moving the penetrating and guiding means 12, i.e. for causing the latter or, primarily, the penetrating and guiding part 18, to penetrate the catheter and/or the membrane 13 and make its way into surrounding glandular tissue, and for being able, after the application of the reference marker 7, to draw said penetrating and guiding part back into the catheter (FIG. 3).

The movement means 14 comprises a flexible movement part 23 which is disposed for movement in the duct 11 in the catheter 9 and which has connected to it a second manoeuvring device 24 by means of which the movement part is caused to move the reference marker 7 along the penetrating and guiding means 12, in the version depicted along its penetrating and guiding part 18, and to drive it into surrounding glandular tissue. In the preferred version, the movement part 23 is with advantage hoselike or tubular and preferably made of plastic material. The second manoeuvring device 24 takes the form of a piston 25 which is disposed for movement in the catheter 9 in the catheter's longitudinal direction, is connected to the movement part 23 and communicates, via a piston rod 26, with a manoeuvring part 27, e.g. a push-button, disposed externally to the catheter, for moving the piston in the catheter, whereby the piston in its turn moves the movement part. Like the manoeuvring part 22, the manoeuvring part 27 obviously remains external to the catheter 9 at the latter's distal end portion outside the urethra, for easy activation or movement of the movement means 14, i.e. to make it possible to drive in the reference marker 7 to an intended position and thereafter be able to draw the movement means, primarily the movement part 23, back into the catheter (FIG. 3).

In the preferred version depicted, the penetrating and guiding part 18 is adapted to running through the hoselike or tubular movement part 23 and is movable in the latter's longitudinal direction, resulting in a compact and elegant combination of said parts 18, 23 (FIGS. 3 and 4). The inside diameter of the movement part 23 is of course somewhat larger than the diameter of about 0.3 to 0.5 mm of the penetrating and guiding part 18.

The piston 25 of the second manoeuvring device 24 is disposed in the longitudinal direction of the catheter 9 nearer to the latter's insertion end than the piston 20 of the first manoeuvring device 19. The piston rod 26 of the piston 25 of the second manoeuvring device 24 runs through the piston rod 21 and the piston 20 of the first manoeuvring device 19 and is movable in the latter piston rod's longitudinal direction. Here again the result is a compact and practical configuration of said manoeuvring devices 19, 24 (FIG. 3).

In the preferred version depicted, the catheter 9 has a plurality of ducts, and one or more reference markers 7 are disposed for movement therein. This makes it possible to use the same catheter 9 for applying two or more reference markers 7 in the prostate glandular tissue on the same occasion. Applying a number of reference markers 7 at known mutual positions makes it easier to determine how the treatment beam 2 is aimed. In the preferred version, the catheter 9 has three ducts 11 and a reference marker 7 is disposed for movement in each duct. Depending on the particular need and application, however, it is of course also possible within the scope of the invention for the catheter 9 to be provided with two or more than three ducts 11 and two or more reference markers 7 disposed for movement therein. The ducts 11 end with advantage at different locations in the longitudinal direction of the catheter 9, at a distance of preferably about 1.0 cm from one another, and lead with advantage radially outwards from the catheter in mutually different directions, in the version with three ducts at mutual angles of preferably about 120°.

With a view to as simple a configuration as possible for the instrument 8 according to the invention, the penetrating and guiding parts 18 in all the ducts 11 have a first manoeuvring device 19 in common, and the movement parts 23 in all the ducts 11 have a second manoeuvring device 24 in common. The result is simultaneous application of all the reference markers 7. For the movement of the various parts 18, 23 to be as problem-free as possible, the manoeuvring devices 19, 24 are in principle disposed coaxially with the central longitudinal axis of the catheter 9, while the penetrating and guiding parts 18 and the movement parts 23 are in principle parallel but not coaxial with the catheter's central longitudinal axis, preferably on, in cross-sectional terms, a circular line round said central longitudinal axis.

The instrument 8 according to the invention has with advantage a stop mechanism 28 which allows activation of the movement means 14 in order to move the reference marker 7 and drive it into surrounding glandular tissue, but only after the penetrating and guiding means 12 has been activated to penetrate the catheter 9 and/or the membrane 13 totally or partly covering the catheter and make its way into the surrounding glandular tissue. The stop mechanism 28 is adapted to locking the manoeuvring part 27 of the second manoeuvring device 24 until the manoeuvring part 22 of the first manoeuvring device 19 has reached an end-position (not depicted) at which the penetrating and guiding part 18 has made its way far enough into surrounding glandular tissue to provide guidance thereafter for the reference marker 7 while it is being driven into the surrounding glandular tissue. According to a depicted preferred version, the stop mechanism 28 takes the form of a sleeve-shaped part which, in order to prevent movement of the manoeuvring part 27 of the second manoeuvring device 24, is disposed about the piston rod 26 of said second manoeuvring device, being preferably clamped between the manoeuvring part 27 and a stop on the distal end portion of the catheter 9, but which is released for removal when the manoeuvring part 22 of the first manoeuvring device 19 has reached said end-position. The sleeve-shaped part 28 therefore prevents the push-button 27 from being pressed in until the handle 22 has reached such a position that the sleeve part is exposed to such an extent that it can be removed. The end-position allowing activation of the manoeuvring part 27 of the second manoeuvring device 24 may be considered to have been reached, for example, when the manoeuvring part 22 of the first manoeuvring device 19 reaches the stop on the distal end portion of the catheter 9 or when the rear piston 20 of the first manoeuvring device 19 reaches the forward piston 25 which forms part of the second manoeuvring device 24 and which is situated nearer to the catheter's insertion end.

It should be noted finally that the catheter 9 preferably comprises not only said duct or ducts 11 for one or more reference markers 7 but also a duct (not depicted) for manoeuvring the fixing means 10.

It should also be noted that in each case of a catheter 9 with a plurality of ducts 11 for one or more reference markers 7, a membrane 13 covering the whole catheter, as in the version depicted, is most advantageous, since this is the easiest version to manufacture. In general, however, any membrane used may also be designed to cover only the part or parts of the catheter 9 to which the duct or ducts 11 lead or simply in principle only the orifice(s) of said duct(s). With or without a membrane, any portion of the sidewall 9a of the catheter 9 which covers the respective duct orifice(s) is preferably thinner and therefore more readily penetrable than other portions of the catheter sidewall. It should also be mentioned that the membrane 13 is with advantage made of latex or silicone.

When the catheter 9 has been introduced into the urethra, inserted through it to a desired location near to the prostate gland and fixed in the urethra, the method for permanent application of a reference marker 7 involves the penetrating and guiding means 12 being caused to penetrate the catheter and/or the membrane 13 totally or partly covering the catheter and being introduced into glandular tissue surrounding the catheter and the urethra. As described above, the penetrating and guiding means 12 is with advantage caused to penetrate a portion of the sidewall 9a of the catheter 9 and/or a side portion 13a of the membrane 13. During the insertion, the penetrating and guiding means 12 is guided on the basis of the reference marker 7.

After the insertion of the penetrating and guiding means 12 into surrounding glandular tissue, the reference marker 7 is moved by the movement means 14 along said penetrating and guiding means and is driven by the movement means into surrounding glandular tissue for permanent application therein. This driving into the glandular tissue takes place alongside the catheter 9 and the urethra. The reference marker 7 is driven about 5 to 15 mm into the glandular tissue. The version depicted in the drawings of the instrument 8 according to the invention may be used to cause a plurality of penetrating and guiding means 12 to penetrate the catheter 9 and/or the membrane 13 and may be inserted in surrounding glandular tissue at various locations in the catheter's longitudinal direction and radially outwards from the catheter in mutually different directions. A plurality of reference markers 7 are thereafter moved along the penetrating and guiding means 12 and are driven into the surrounding glandular tissue at the same locations and in the same directions as the penetrating and guiding means. Where there are three penetrating and guiding means 12, they are caused, as indicated above, to penetrate the catheter 9 and/or the membrane 13 and are inserted in surrounding glandular tissue at mutual distances of preferably about 1.0 cm in the catheter's longitudinal direction and radially outwards from the catheter at mutual angles of preferably about 120°, followed by three reference markers 7 being moved along the penetrating and guiding means and being driven into the glandular tissue at in principle the same distances from one another in the catheter's longitudinal direction and radially outwards from the catheter in principle at the same mutual angles as the penetrating and guiding means. This involves all the penetrating and guiding means 12 and all the reference markers 7 being respectively activated and moved simultaneously.

After the application of the reference marker or markers 7, the movement means 14 and penetrating and guiding means 12 are drawn back to their original position inside the catheter 9, followed by the catheter being drawn out from the urethra.

Although the instrument according to the invention is described above in connection with permanent application of reference markers in prostate glandular tissue, it is possible within the scope of the invention to permanently apply, via a body cavity, body orifice etc., at least one reference marker in tissue situated around the body cavity, body orifice etc. in order by means of the marker to allow precision aiming of a treatment beam at a cancer tumour in that tissue.

It will be obvious to one skilled in the art that the present invention may be modified and altered within the scope of the claims set out below without departing from the idea and objects of the invention. Thus it has probably been indicated above that, for example, constituent parts of the instrument may vary in shape, number and positioning, depending on, for example, the application, how it is desired to apply the reference marker or markers and, in the case of a plurality of reference markers, whether it is desired to do so simultaneously or perhaps one at a time. The choice of materials for the various parts may also vary, depending on the latter's intended characteristics.

1: A method for permanently applying via the urethra at least one reference marker in the prostate gland in order by means of the marker to allow precision aiming of a treatment beam at a cancer tumor wherein,
a catheter (9) with at least one duct (11) running in principle in the catheter's longitudinal direction, and at least one reference marker (7) disposed for movement in the duct, is introduced into the urethra and inserted through it to the prostate gland,
a means (12), with guidance by the reference marker (7), is caused, from the duct (11), to penetrate the catheter (9) and/or a membrane (13) totally or partly covering the catheter and is inserted in glandular tissue surrounding the catheter and the urethra, and
the marker (7) in said duct (11) is thereafter moved by a means (14) along said penetrating and guiding means (12) and is driven by said movement means, with guidance by the penetrating and guiding means, into the glandular tissue surrounding the catheter (9) and the urethra, for permanent application therein.

2: A method according to claim 1, wherein said penetrating and guiding means (12) is caused to penetrate a portion of the sidewall (9a) of the catheter (9) and/or a side portion (13a) of the membrane (13) totally or partly covering the catheter, and that the reference marker (7) is moved along the penetrating and guiding means and is driven into surrounding glandular tissue alongside the catheter and the urethra by said movement means (14).

3: A method according to claim 1, wherein a plurality of penetrating and guiding means (12) are caused to penetrate the catheter (9) and/or the membrane (13) totally or partly covering the catheter and are inserted in surrounding glandular tissue at various locations in the catheter's longitudinal direction and radially outwards from the catheter in mutually different directions, and that a plurality of reference markers (7) are thereafter moved along said penetrating and guiding means and are driven into the surrounding glandular tissue at the same locations and in the same directions as the penetrating and guiding means.

4: A method according to claim 3, wherein three penetrating and guiding means (12) are caused to penetrate the catheter (9) and/or the membrane (13) totally or partly covering the catheter and are inserted in surrounding glandular tissue at mutual distances of preferably about 1.0 cm in the catheter's longitudinal direction and radially outwards from the catheter at mutual angles of preferably about 120°, and that three reference markers (7) are thereafter moved along said penetrating and guiding means and are driven into the surrounding glandular tissue at in principle the same distances from one another in the catheter's longitudinal direction and radially outwards from the catheter at in principle the same mutual angles as the penetrating and guiding means.

5: A method according to claim 3, wherein all the penetrating and guiding means (12) are caused to penetrate the catheter (9) and/or the membrane (13) totally or partly covering the catheter and are inserted in surrounding glandular tissue simultaneously, and that all the reference markers (7) are thereafter moved along said penetrating and guiding means and are driven into the surrounding glandular tissue simultaneously.

6: A method according to claim 1, wherein the catheter (9) is fixed in the urethra after insertion in the urethra but before the activation of the penetrating and guiding means (12).

7: A method according to claim 1, wherein after the application of the reference marker (7) said movement means (14) and said penetrating and guiding means (12) are drawn back to their original positions, followed by the catheter (9) being drawn out from the urethra.

8: An instrument for implementing the method according to claim 1 for permanently applying via the urethra at least one reference marker in the prostate gland in order by means of the marker to allow precision aiming of a treatment beam at a cancer tumor wherein,
a catheter (9) insertable via the urethra to the prostate gland, with at least one duct (11) running in principle in the catheter's longitudinal direction,
at least one reference marker (7) disposed for movement in the duct (11),
a means (12) designed, with guidance by the reference marker (7) to, from the duct (11), penetrate the catheter (9) and/or the membrane (13) totally or partly covering the catheter, make its way into glandular tissue surrounding the catheter and the urethra and guide the reference marker (7) during the latter's movement, and
a means (14) designed to move the reference marker (7) along said penetrating and guiding means (12) and, with guidance by the penetrating and guiding means, to drive the marker into glandular tissue surrounding the catheter (9) and the urethra, for permanent application therein.

9: An instrument according to claim 8, wherein the duct (11) comprises an end portion (11a) which is directed towards a portion of the sidewall (9a) of the catheter (9) and/or a side portion (13a) of the membrane (13) totally or partly covering the catheter and which ends immediately within said portion of the catheter sidewall and/or immediately within said side portion of the membrane covering the catheter.

10: An instrument according to claim 9, wherein the reference marker (7) is disposed for movement in said end portion (11a) of the duct (11).

11: An instrument according to claim 8 wherein the reference marker (7) is designed, preferably with a duct (15) running through it in its longitudinal direction, to guide the penetrating and guiding means (12) when the latter penetrates the catheter (9) and/or the membrane (13) totally or partly covering the catheter and makes its way into surrounding glandular tissue, and thereafter via the penetrating and guiding means, to guide the marker when the latter is being driven into the surrounding glandular tissue.

12: An instrument according to claim 11, wherein said duct (15) runs coaxially with the central longitudinal axis of the reference marker (7).

13: An instrument according to claim 8, wherein the reference marker (7) has a recess (16) at one end for said movement means (14) and narrows at its opposite end to a tip (17) to make it easier to drive into surrounding glandular tissue.

14: An instrument according to claim 8, wherein said penetrating and guiding means (12) comprises a flexible penetrating and guiding part (18) which is arranged for movement in the duct (11) in the catheter (9) and which has connected to it a first maneuvering device (19) by means of which the penetrating and guiding part is caused to penetrate the catheter and/or the membrane (13) totally or partly covering the catheter and to make its way into the glandular tissue surrounding the catheter and the urethra.

15: An instrument according to claim 14, wherein the penetrating and guiding part (18) takes the form of a thin metal wire with a pointed front end (18a) and a diameter of about 0.3 to 0.5 mm.

16: An instrument according to claim 14, wherein said first maneuvering device (19) takes the form of a piston (20) which is disposed for movement in the catheter (9) in the latter's longitudinal direction, is connected to the penetrating and guiding part (18) and communicates via a piston rod (21) with a maneuvering part (22), preferably a handle, disposed externally to the catheter, for moving the piston in the catheter.

17: An instrument according to claim 8, wherein said movement means (14) comprises a flexible movement part (23) which is arranged for a movement in the duct (11) in the catheter (9) and which has connected to it a second maneuvering device (24) by means of which the movement part is caused to move the reference marker (7) along the penetrating and guiding means (12) and to drive it into surrounding glandular tissue.

18: An instrument according to claim 17, wherein the movement part (23) takes the form of a hoselike or tubular component preferably made of plastic material.

19: An instrument according to claim 17, wherein said second maneuvering device (24) takes the form of a piston (25) which is disposed for movement in the catheter (9) in the catheter's longitudinal direction, is connected to the movement part (23) and communicates via a piston rod (26) with a maneuvering part (27), preferably a pushbutton, disposed externally to the catheter, for moving the piston in the catheter.

20: An instrument according to claim 18, wherein the penetrating and guiding part (18) runs through the movement part (23) and is movable in the latter's longitudinal direction.

21: An instrument according to claim 19, wherein the piston (25) of said second maneuvering device (24) is disposed in the longitudinal direction of the catheter (9) nearer to the latter's insertion end than the piston (20) of said first maneuvering device (19), and that the piston rod (26) of the piston of said second maneuvering device is adapted to running through the piston rod (21) and the piston (20) of said first maneuvering device and is movable in the longitudinal direction of that piston rod (21).

22: An instrument according to claim 8, wherein the catheter (9) has a plurality of ducts (11) and that one or more reference markers (7) are disposed for movement therein.

23: An instrument according to claim 22, wherein the catheter (9) has three ducts (11) and that a reference marker (7) is disposed for movement in each duct.

24: An instrument according to claim 22, wherein the ducts (11) end at different locations in the longitudinal direction of the catheter (9) and lead radially outwards from the catheter in mutually different directions.

25: An instrument according to claim 24, wherein three ducts (11) end at mutual distances of preferably about 1.0 cm in the longitudinal direction of the catheter (9) and lead radially outwards from the catheter at mutual angles of preferably about 120°.

26: An instrument according to claim 22, wherein a first maneuvering means (19) common to the penetrating and guiding parts (18) in all the ducts (11), and by a second maneuvering device (24) common to the movement parts (23) in all the ducts (11).

27: An instrument according to claim 26, wherein said first and second maneuvering devices (19, 24) are disposed coaxially with the central longitudinal axis of the catheter (9) and that the penetrating and guiding parts (18) and the movement parts (23) run in principle parallel but not coaxially with the central longitudinal axis of the catheter.

28: An instrument according to claim 8, wherein a stop mechanism (28) which allows activation of the movement means (14) for moving the reference marker (7) and driving it into surrounding glandular tissue, but only after the penetrating and guiding means (12) has been activated to penetrate the catheter (9) and/or the membrane (13) totally or partly covering the catheter and make its way into the surrounding glandular tissue, tissue.

29: An instrument according to claim 28, wherein the stop mechanism (28) is designed to lock the maneuvering part (27) of said second maneuvering device (24) until the maneuvering part (22) of said first maneuvering device (19) has reached an end position at which the penetrating and guiding part (18) makes its way far enough into surrounding glandular tissue to thereafter provide guidance for the reference marker (7) during the driving of the latter into the surrounding glandular tissue.

30: An instrument according to claim 29, wherein the stop mechanism takes the form of a sleeve-shaped part which, in order to prevent movement of the maneuvering part (27) of the second maneuvering device (24), is disposed about the piston rod (26) of the second maneuvering device, but which is released for removal when the maneuvering part (22) of the first maneuvering device (19) has reached said end position.

31: An instrument according to claim 8, wherein a means (10) for fixing the catheter (9) in the urethra.

32: An instrument according to claim 31, wherein the catheter (9) has in the longitudinal direction not only said at least one duct (11) for at least one reference marker (7) but also a duct for maneuvering the fixing means (10).

33: An instrument according to claim 8, wherein the membrane (13) totally or partly covering the catheter 9 is made of latex or silicone.

34: Use of an instrument according to claim 8 for permanently, via a body cavity, body orifice etc., applying at least one reference marker in tissue situated around the body cavity, body orifice etc in order by means of the marker to allow precision aiming of a treatment beam at a cancer tumour in that tissue.
